(19) United States
(12) Patent Application Publication
(10) Pub. No.: US 2019/0381132 A1
(43) Pub. Date: Dec. 19, 2019

(54) METHODS AND COMPOSITIONS FOR TREATMENT OF IONIZING RADIATION RESISTANT TUMORS

(71) Applicant: Shenzhen International Institute for Biomedical Research, Shenzhen (CN)

(72) Inventors: Minfeng Shu, Shenzhen (CN); Stephen Mallon, Shenzhen (CN); Ralph R. Weichselbaum, Guangdong (CN); Bernard Roizman, Shenzhen (CN)

(21) Appl. No.: 16/488,101

(22) PCT Filed: May 17, 2017
(86) PCT No.: PCT/CN2017/084712

§ 371 Date: Aug. 22, 2019

(51) Int. Cl. A61K 38/17; C12N 15/113; A61K 31/12; A61K 31/57; A61K 31/05; A61K 31/085; C12N 7/00; C07K 14/47

(52) U.S. Cl. CPC A61K 38/1709; C12N 15/113; A61K 31/12; A61K 31/57; A61K 31/05; A61K 48/00; C12N 7/00; C07K 14/47; C12N 2310/11; C12N 2740/15043; C12N 2310/14; A61K 31/085

(57) ABSTRACT

Disclosed is a method for treatment of a tumor in a subject comprising administering to the subject a therapeutically effective amount of an ATF3 activator. Use of an ATF3 activator in the preparation of a pharmaceutical composition for treatment of a tumor in a subject is also disclosed.

### TECHNICAL FIELD

The present invention concerns methods and compositions for treating tumors resistant to ionizing radiation. The invention provides methods and compositions for altering properties of cellular signaling networks in tumors cells. In certain embodiments the methods and compositions involve activating transcription factor 3 (ATF3), a variant thereof or a pharmaceutical agent and composition directed thereto.

### BACKGROUND

Radiotherapy, the exposure of tumors to therapeutic doses of ionizing radiation, is delivered with curative and palliative intent to 60% of all cancer patients. Approximately 600,000 patients receive radiotherapy in the United States. Patients with localized head and neck, CNS, lung, breast, GI (esophageal, pancreatic, rectal), GU (prostate, bladder), lymphoma, skin and soft tissue sarcomas and pediatric tumors receive radiotherapy as an alternative to surgery in early stage disease and in combination with surgery and chemotherapy during late stage disease. As such, along with chemotherapy and surgery, radiotherapy is a key cancer treatment modality.

Radioresistance is the term used to describe cancer cells that survive therapeutic doses of ionizing radiation. The causes of radioresistance are incompletely understood but are thought to stem from a complex interplay between properties of the cancer cell and its environment. Proposed mechanisms include increased expression of free radical scavengers such as glutathione, alterations to DNA repair mechanisms, and suppression of effector cells of the immune system.

ATF3 (Activating Transcriptional Factor 3) belongs to the ATF/cyclic AMP response element binding (ATF/CREB) transcription factor family that contains proteins of diverse size, sequence and biological function. The family includes ATF1 (also known as TREB36), CREB, CREM, ATF2 (also known as CRE-BP1), ATF3, ATF4, ATF5 (also known as ATFX), ATF6, ATF7, and B-ATF. The common feature that these proteins share is the bZIP element. The bZIP element contains a region enriched in amino acids with basic charge and a leucine zipper region. The basic region in this domain determines DNA binding specificity, while the leucine zipper region is required for interactions with other proteins containing bZIP elements. ATF/CREB proteins were initially identified as proteins that bound the cyclic AMP response element (CRE) in various promoters. The CRE response element contains the consensus sequence TGACGTCA. In contrast to other members of the ATF/CREB family, ATF3 expression is rapidly induced by a wide range of cellular stresses including nutrient deprivation, oxidative stress, DNA damage, infection by intracellular pathogens and as described above, ionizing radiation. ATF3 has also been reported to be induced by small molecules. The small molecules reported to induce ATF3 include curcumin, non-steroidal anti-inflammatory drugs (NSAIDs), progesterone, the phosphatidylinositol inhibitors LY294002 and tert-butylhydroquinone (tBHQ) and butylated hydroxyanisole (BHA). ATF3 interacts numerous proteins to effect processes related to cell survival and death.

### SUMMARY

In the present invention expression of ATF3 in select cancer lines induces death or suppression of proliferation. In certain embodiments death may occur even in the absence of IR. The scientific model is as follows. Cancer cells derived from tumors that are sensitive to IR express ATF3 which signals to downstream effector proteins. In cells that are derived from tumors that are resistant to IR ATF3 is not expressed or it cellular function is impaired. When forced to express ATF3 such radioresistant cells will die in a manner that is independent of ionizing radiation.

In one aspect of the invention, a method for treatment of a tumor that is resistant to ionizing radiation in a subject is provided. The method comprises administering to the subject in need of treatment a therapeutically effective amount of an ATF3 activator. In some embodiments, the ATF3 activator is a small molecule able to induce ATF3 expression, for example curcumin, non-steroidal anti-inflammatory drugs, progesterone, a phosphatidylinositol inhibitor, tert-butylhydroquinone (tBHQ) and butylated hydroxyanisole (BHA). In some embodiments, the ATF3 activator is a vector carrying a polynucleotide encoding ATF3 protein, including viral and non-viral vectors encoding ATF3 protein. In some embodiments, the ATF3 activator is a vector carrying a non-coding RNA directed to an inhibitor of ATF3 expression, such as an exosome carrying an antisense oligomer directed to an inhibitor of ATF3 expression. In some embodiments, the tumor does not express ATF3 protein. In some embodiments, the tumor does express ATF3 protein.

In some embodiments, the administration of the ATF3 activator results in expression of the ATF3 protein in a cell of the tumor. In some embodiments, the administration of the ATF3 activator leads to an elevated expression of ATF3 in a cell of the tumor. In some embodiments, the administration of the ATF3 activator causes accumulation of ATF3 protein in a cell of the tumor. In some embodiments, the administration of the ATF3 activator causes tumor cell death.

In another aspect of the invention, provided is use of an ATF3 activator in the preparation of a pharmaceutical composition for treatment of an ionizing radiation resistant tumor in a subject. In some embodiments, the ATF3 activator is a small molecule capable to induce ATF3 expression, for example curcumin, non-steroidal anti-inflammatory drugs, progesterone, a phosphatidylinositol inhibitor, tert-butylhydroquinone (tBHQ) and butylated hydroxyanisole (BHA). In some embodiments, the ATF3 activator is a vector carrying a polynucleotide encoding ATF3 protein, including viral and non-viral vectors encoding ATF3 protein. In some embodiments, the ATF3 activator is a vector carrying a non-coding RNA directing to an ATF3 expression inhibitor, such as an exosome carrying an antisense oligomer directed to an ATF3 expression inhibitor. In some embodiments, the tumor does not express ATF3 protein. In some embodiments, the tumor does express ATF3 protein.

In another aspect of the invention, an artificial vector for treatment of an ionizing radiation resistant tumor in a subject, wherein the vector carries a polynucleotide encoding ATF3 protein, is provided. In some embodiments, the artificial vector is a viral vector encoding ATF3 protein. In some embodiments, the viral vector is derived from a lentivirus, an adeno-associated virus or a herpes-simplex virus. In some embodiments, the tumor does not express ATF3 protein. In some embodiments, the tumor does express ATF3 protein.

In another aspect of the invention a pharmaceutical composition containing an ATF3 activator for treatment of a tumor resistant to ionizing radiation in a subject is provided. In some embodiments, the pharmaceutical composition comprises a therapeutically effective amount of an ATF3 activator and a pharmaceutically acceptable excipient. In some embodiments the ATF3 activator is a small molecule able to induce ATF3 expression including, for example, curcumin, non-steroidal anti-inflammatory drugs (NSAIDs), progesterone, a phosphatidylinositol inhibitor, tertbutylhydroquinone (tBHQ) and butylated hydroxyanisole (BHA). In some embodiments the ATF3 activator is a viral or non-viral vector carrying a polynucleotide encoding ATF3 protein or variants thereof. Therefore, in some embodiments, the pharmaceutical composition comprises a viral or non-viral gene delivery vector as identified in the present invention and a pharmaceutically acceptable excipient. In some embodiments the ATF3 activator is a non-coding RNA or antisense oligomer directed to an ATF3 expression inhibitor. Therefore, in some embodiments the composition comprises a viral or non-viral vector suitable for the delivery of a non-coding RNA or oligonucleotide and a pharmaceutically acceptable excipient.

### BRIEF DESCRIPTION OF DRAWINGS

FIG. 1. Survival of IR resistant and IR sensitive cell lines following exposure to doses of IR ranging from 0 to 8 Gy. The procedures were the same for all cell lines. Briefly, the cells were seeded to form colonies in 6-well plates and treated the next day with an increasing amount of IR. When sufficiently large colonies with at least 50 cells were visible (12-15 d), the plates were fixed with methanol and stained with crystal violet. Colonies with more than 50 cells were counted and the surviving fraction was calculated using generally accepted methods. Cells were divided into two groups based on their survival fractions: Cells (HCT116, D54, U251MG) treated with 8 Gy with surviving fraction ≤0.05 were named sensitive cells; Cells (Du145, Widr, T24) with surviving fraction ≥0.5 were named IR resistant cells.

FIG. 2. Following exposure to IR ATF3 protein is induced in radiosensitive cell lines but not radioresistant cell lines. Radiosensitive cells (U251MG, D54, HCT116 (Panel A) and radioresistant cells (T24, Du145, Widr (Panel B) were mock treated or treated with 6 Gy. At indicated times the cells were collected, and equal amounts of total proteins were separated electrophoretically on a 12.5% denaturing polyacrylamide gel, transferred to a nitrocellulose sheet, and probed with the mouse monoclonal antibody to β-actin and rabbit polyclonal antibody to ATF3.

FIG. 3. Depletion of ATF3 causes sensitive cells to become resistant to IR. ATF3 was depleted in IR sensitive cells (HCT116) using CRISPR/cas9 technology as described in Materials and Methods. Panel A: Production of ATF3 in clone Scr, clone 7 and clone 8 cells was detected by immunoblotting. Panel B: HCT116 cells from which ATF3 was depleted were exposed to ionizing radiation at a dose of 6Gy. The viability of the cells was measured using the MTT assay.

FIG. 4. Forced expression of ATF3 kills radioresistant cells in the absence of IR. Panel A: Accumulation of ATF3 in Du145 cells stably expressing ATF3. The cell line was transformed with a lentivirus expressing ATF3 tagged with FLAG epitope at N-terminal and selected by puromycin. ATF3 was detected by immunoblotting. The effects of ATF3 on cell viability was detected by MTT assay (Panel C) and colony formation assay (Panel B). Quantification of the relative cell viability of the colony formation is shown in Panel D.

FIG. 5. ATF3 protein is induced by HSV-1 infection in radioresistant cells. Du145 cells (Panel A) and Widr cells (Panel B) were mock infected or exposed to 10 PFU of HSV-1(F) per cells. Cells were collected at indicated times post infection. To measure expression of ATF3 equal amounts of total proteins were separated electrophoretically on a 12.5% denaturing polyacrylamide gel, transferred to a nitrocellulose sheet, and probed with the mouse monoclonal antibody to β-actin, mouse polyclonal antibody to ICP8 and rabbit polyclonal antibodies to ATF3.

### DETAILED DESCRIPTION

### Definitions

It is to be noted that the term “a” or “an” entity refers to one or more of that entity; for example, “an ATF3 activator,” is understood to represent one or more ATF3 activator. As such, the terms “a” (or “an”), “one or more,” and “at least one” can be used interchangeably herein.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on. The subject herein is preferably a human.

As used herein, phrases such as “to a patient in need of treatment” or “a subject in need of treatment” includes subjects, such as mammalian subjects, that would benefit from administration of an ATF3 activator or composition of the present disclosure used, e.g., for detection, for a diagnostic procedure and/or for treatment.

As used herein, phrases such as “an ionizing radiation resistant tumor”, “an IR resistant tumor” “a radiation resistant tumor”, “a tumor resistant to ionizing radiation” or “a resistant tumor” refers to a tumor or a cancer showing very low sensitivity to treatment with radiation so that the symptoms thereof are not improved, relived, alleviated, or treated by the radiation treatment. The IR resistant tumor can be a tumor originally resistant to treatment with radiation. Alternatively, the IR resistant tumor can be a tumor not originally resistant, but is no longer sensitive to radiation because a gene in the tumor cells is mutated due to long-term administration of the radiation or is otherwise resistant. In the present invention, the resistant tumor may be any tumor showing resistance to radiation treatment, but is not specifically limited thereto.

As used herein, the term “tumor” refers to a malignant tissue comprising transformed cells that grow uncontrollably (i.e., is a hyperproliferative disease). Tumors include leukemias, lymphomas, myelomas, plasmacytomas, and the like; and solid tumors. Examples of solid tumors that can be treated according to the invention include but are not limited to sarcomas and carcinomas such as melanoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma.

The term “ATF3 activator” as used herein refers to a chemical or biological agent capable to induce or introduce the expression of ATF3 protein in an IR resistant tumor cell. The present invention has demonstrated that the accumulation of ATF3 protein in an IR resistant tumor cell ultimately causes tumor cell death. By “induce” it is meant that some ATF3 activators activate the expression of ATF3 protein by making use of the Atf3 encoding gene encoded in the tumor cell. Induction causes initiation of expression and subsequently or concurrently accumulation of ATF3 protein in the tumor cell and ultimately cell death. By “introduce” it is meant some activators introduce to the tumor cell heterogeneous polynucleotides encoding the ATF3 protein so that the heterogeneous polynucleotide encoding the ATF3 protein is stably expressed in the tumor cell. The expression of the heterogeneous polynucleotide encoding the ATF3 protein, alone or in combination with the baseline expression, if any, of ATF3 protein in the tumor cell, results in accumulation of the ATF3 protein in the tumor cell and ultimately causes cell death.

The term “therapeutically effective amount” or “pharmaceutically effective amount” as used in this specification refers to an amount of each active ingredient that can exert clinically significant effects. The pharmaceutically effective amount of the ATF3 activator for a single dose may be prescribed in a variety of ways, depending on factors such as formulation methods, administration manners, age of patients, body weight, gender, pathologic conditions, diets, administration time, administration interval, administration route, excretion speed, and reaction sensitivity. For example, the pharmaceutically effective amount of the ATF3 activator for a single dose may be in ranges of 0.001 to 100 mg/kg, or 0.02 to 10 mg/kg, but not limited thereto. The pharmaceutically effective amount for the single dose may be formulated into a single formulation in a unit dosage form or formulated in suitably divided dosage forms, or it may be manufactured to be contained in a multiple dosage container.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer.

The present invention employs, among others, antisense oligomer and similar species for use in modulating the function or effect of nucleic acid molecules encoding an inhibitor of ATF3 protein. An inhibitor of ATF3 protein as used herein is defined as a molecule that blocks or reduces the expression of ATF3 protein by interfering with transcription of the ATF3 gene, processing or translation of ATF3 mRNA or stability of ATF3 protein. The inhibitor can be any molecule involved in the cascade of a pathway inhibiting ATF3 expression in an IR resistant tumor cell. This is accomplished by providing oligonucleotides which specifically hybridize with one or more nucleic acid molecules encoding the inhibitor. As used herein, the terms “target nucleic acid” and “nucleic acid molecule encoding the inhibitor” have been used for convenience to encompass DNA encoding the inhibitor, RNA (including pre-mRNA and mRNA or portions thereof) transcribed from such DNA or synthesized de novo and also cDNA derived from such RNA. An oligomer of this invention that hybridizes with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of the inhibitor. In the context of the present invention, “modulation” and “modulation of expression” mean decrease in the amount or levels of a nucleic acid molecule encoding the gene, e.g., DNA or RNA. mRNA is often a preferred target nucleic acid.

In the context of this invention, “hybridization” means the pairing of complementary strands of oligomers. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense oligomer is specifically hybridizable when binding of the oligomer to the target nucleic acid interferes with the normal function of the target nucleic acid to cause a loss of activity, and there is a sufficient degree of complementarity to avoid non-specific binding of the antisense oligomer to non-target nucleic acid sequences under conditions in which specific binding is desired, i.e., under physiological conditions in the case of in vivo assays or therapeutic treatment, and under conditions in which assays are performed in the case of in vitro assays.

It is understood in the art that the sequence of an antisense oligomer need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the antisense compounds of the present invention comprise at least 70%, or at least 75%, or at least 80%, or at least 85% sequence complementarity to a target region within the target nucleic acid, more preferably that they comprise at least 90% sequence complementarity and even more preferably comprise at least 95% or at least 99% sequence complementarity to the target region within the target nucleic acid sequence to which they are targeted. For example, an antisense compound in which 18 of 20 nucleobases of the antisense oligomer are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense oligomer which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

### ATF3 Activators

The present invention relates to methods and compositions involved with use of ATF3 activators. An ATF3 activator is defined as a chemical or biological agent capable to induce or introduce the expression of ATF3 protein in an IR resistant tumor cell.

Some activators such as small molecules activate the expression of ATF3 protein making use of the gene encoding ATF3 originally existing in the tumor cell. Examples of such ATF3 activators include small molecules exemplified by curcumin, non-steroidal anti-inflammatory drugs, progesterone, the phosphatidylinositol inhibitor, tert-butylhydroquinone (tBHQ) and butylated hydroxyanisole (BHA), and vectors carrying antisense oligonucleotides directed to an ATF3 expression inhibitor. The latter example is useful in the circumstance that the expression of ATF3 in an IR resistance tumor cell is inhibited by an inhibitor known in the art.

Alternatively, some activators introduce heterogeneous polynucleotide encoding the ATF3 protein to the tumor cell so that the heterogeneous polynucleotide encoding the ATF3 protein is stably expressed in the tumor cell, resulting in accumulation of the ATF3 protein in the tumor cell and, after subsequent interactions, causing tumor cell death. ATF3 activators of this kind include artificial delivery systems carrying ATF3 encoding polynucleotides including both viral and non-viral vectors.

### Viral Vectors

In one embodiment of the invention, an ATF3 activator is a viral vector carrying an ATF3 encoding polynucleotide. A viral vector may also be called a vector, vector virion or vector particle. In another embodiment, the viral vector is derived from a retrovirus, adenovirus, adeno-associated virus, herpes simplex virus, vaccinia virus or baculovirus.

The retroviral vector of the present invention may be derived from or may be derivable from any suitable retrovirus. A large number of different retroviruses have been identified. Examples include: murine leukemia virus (MLV), human T-cell leukemia virus (HTLV), mouse mammary tumor virus (MMTV), Rous sarcoma virus (RSV), Fujinami sarcoma virus (FuSV), Moloney murine leukemia virus (Mo MLV), FBR murine osteosarcoma virus (FBR MSV), Moloney murine sarcoma virus (Mo-MSV), Abelson murine leukemia virus (A-MLV), Avian myelocytomatosis virus-29 (MC29) and Avian erythroblastosis virus (AEV). A detailed list of retroviruses may be found in Coffin et al. (1997) “Retroviruses”, Cold Spring Harbor Laboratory Press Eds: J M Coffin, S M Hughes, H E Varmus pp 758-763. In another embodiment, the retrovirus is derived from a foamy virus.

Lentiviruses are part of a larger group of retroviruses. A detailed list of lentiviruses may be found in Coffin et al (1997) “Retroviruses” Cold Spring Harbor Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV) and bovine immunodeficiency virus (BIV). A lentiviral vector, as used herein, is a vector which comprises at least one component part derivable from a lentivirus. Preferably, that component part is involved in the biological mechanisms by which the vector infects cells, expresses genes or is replicated. The lentiviral vector may be derived from either a primate lentivirus (e.g. HIV-1) or a non-primate lentivirus. Examples of non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV). In another embodiment, the lentiviral vector is derived from HIV-1, HIV-2, SIV, FIV, BIV, EIAV, CAEV or visna lentivirus.

In another embodiment of the present invention, the viral vector may be an adenovirus vector. The adenovirus is a double-stranded, linear DNA virus that does not replicate through an RNA intermediate. Adenoviruses are double-stranded DNA non-enveloped viruses that are capable of in vivo, ex vivo and in vitro transduction of a broad range of cell types of human and non-human origin. These cells include respiratory airway epithelial cells, hepatocytes, muscle cells, cardiac myocytes, synoviocytes, primary mammary epithelial cells and post-mitotically terminally differentiated cells such as neurons. Adenoviruses have been used as vectors for gene therapy and for expression of heterologous genes. The large (36 kb) genome can accommodate up to 8 kb of foreign insert DNA and is able to replicate efficiently in complementing cell lines to produce very high titers of up to 10¹² transducing units per ml. Adenovirus is thus one of the best systems to study the expression of genes in primary non-replicative cells. The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they exist as an episome (independently from the host genome) as a linear genome in the host nucleus.

Adeno-associated virus (AAV) is an attractive vector system for use in the present invention as it has a high frequency of integration and it can infect non-dividing cells. This makes it useful for delivery of genes into mammalian cells. AAV has a broad host range for infectivity. Details concerning the generation and use of rAAV vectors are described in U.S. Pat. Nos. 5,139,941 and 4,797,368, each incorporated herein by reference. Recombinant AAV vectors have been used successfully for in vitro, ex vivo and in vivo transduction of marker genes and genes involved in human diseases. Certain AAV vectors have been developed which can efficiently incorporate large payloads (up to 8-9 kb). One such vector has an AAV5 capsid and an AAV2 ITR (Allocca M, et al J. Clin Invest (2008) 118: 1955-1964).

Herpes simplex virus (HSV) is an enveloped double-stranded DNA virus that naturally infects neurons. It can accommodate large sections of foreign DNA, which makes it attractive as a vector system, and has been employed as a vector for gene delivery to neurons (Manservigiet et al Open Virol J. (2010) 4:123-156). The use of HSV in therapeutic procedures requires the strains to be attenuated so that they cannot establish a lytic cycle. In particular, if HSV vectors are used for gene therapy in humans, the polynucleotide should preferably be inserted into an essential gene. This is because if a viral vector encounters a wild-type virus, transfer of a heterologous gene to the wild-type virus could occur by recombination. However, if the recombinant virus is constructed in a way to prevent its replication, this could be accomplished by inserting the oligonucleotide into a viral gene that is essential for replication.

The viral vector of the present invention may be a vaccinia virus vector such as MVA or NYVAC. Alternatives to vaccinia vectors include avipox vectors such as fowlpox or canarypox known as ALVAC and strains derived therefrom which can infect and express recombinant proteins in human cells but are unable to replicate. It is to be appreciated that portions of the viral genome may remain intact following insertion of the recombinant gene. An implication of this is the notion that the viral vector may retain the capacity to infect a cell and subsequently express additional genes that support its replication and possibly promote lysis and death of the infected cell.

A recombinant gene encoding the AFT3 protein contains nucleic acids encoding a protein along with regulatory elements for protein expression. Generally, the regulatory elements that are present in a recombinant gene are selected on the basis of the host cells to be used for expression. Such elements are typically operably-linked to the nucleic acid sequence to be expressed and include a transcriptional promoter, a ribosome binding site, and a terminator. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the virus is introduced into the host cell). The term “regulatory sequence” is intended to include promoters, enhancers and other expression control elements (e.g., polyadenylation signals). Regulatory sequences include those that direct constitutive expression of a nucleotide sequence in many types of host cell and those that direct expression of the nucleotide sequence only in certain host cells (e.g., tissue-specific regulatory sequences).

A newly found regulatory sequence is insulator which includes a class of DNA elements found on cellular chromosomes that protect genes in one region of a chromosome from the regulatory influence of another region. Amelio et al. found a 1.5-kb region containing a cluster of CTCF motifs in the LAT region possesses insulator activities, specifically, enhancer blocking and silencing (Amelio et al. A Chromatin Insulator-Like Element in the Herpes Simplex Virus Type 1 Latency-Associated Transcript RegionBinds CCCTC-Binding Factor and Displays Enhancer-Blocking and Silencing Activities. Journal of Virology, Vol. 80, No. 5, March 2006, p. 2358-2368).

A promoter is defined as a DNA sequence that directs RNA polymerase to bind to DNA and initiate RNA synthesis. A strong promoter is one which causes mRNAs to be expressed to high levels. A suitable element for processing in eukaryotic cells is a polyadenylation signal. Introns may also be present in the gene. Examples of expression cassettes for genes or gene fragments are well known in art.

Appropriate regulatory elements can be selected by those of skill in the art based on, for example, the desired tissue-specificity and level of expression. For example, a cell-type specific or tumor-specific promoter can be used to limit expression of a gene product to a specific cell type. In addition to using tissue-specific promoters, local administration of the viruses can result in localized expression and effect. Examples of non-tissue specific promoters that can be used include the early Cytomegalovirus (CMV) promoter (U.S. Pat. No. 4,168,062) and the Rous Sarcoma Virus promoter. Also, HSV promoters, such as HSV-1 IE and IE 4/5 promoters, can be used. In some embodiments, the promoter is selected from a promoter in following table.

| Promoter | Tumor or Tissue Target |
|---|---|
| B-myb | Glioma liver metastasis |
| Nestin | Glioma |
| CEA (Carinoembryonic antigen) | Colon Cancer |
| Albumin | Hepatoma |
| DF3/MUC1 (Mucin 1) | Pancreatic Cancer |
| Caponin | Leiomyosarcoma |

Examples of tissue-specific promoters that can be used in the technology include, for example, the prostate-specific antigen (PSA) promoter, which is specific for cells of the prostate; the desmin promoter, which is specific for muscle cells; the enolase promoter, which is specific for neurons; the beta-globin promoter, which is specific for erythroid cells; the tau-globin promoter, which is also specific for erythroid cells; the growth hormone promoter, which is specific for pituitary cells; the insulin promoter, which is specific for pancreatic beta cells; the glial fibrillary acidic protein promoter, which is specific for astrocytes; the tyrosine hydroxylase promoter, which is specific for catecholaminergic neurons; the amyloid precursor protein promoter, which is specific for neurons; the dopamine beta-hydroxylase promoter, which is specific for noradrenergic and adrenergic neurons; the tryptophan hydroxylase promoter, which is specific for serotonin/pineal gland cells; the choline acetyltransferase promoter, which is specific for cholinergic neurons; the aromatic L-amino acid decarboxylase (AADC) promoter, which is specific for catecholaminergic/5-HT/D-type cells; the proenkephalin promoter, which is specific for neuronal/spermatogenic epididymal cells; the reg (pancreatic stone protein) promoter, which is specific for colon and rectal tumors, and pancreas and kidney cells; and the parathyroid hormone-related peptide (PTHrP) promoter, which is specific for liver and cecum tumors, and neurilemoma, kidney, pancreas, and adrenal cells.

Examples of promoters that function specifically in tumor cells include the stromelysin 3 promoter, which is specific for breast cancer cells; the surfactant protein A promoter, which is specific for non-small cell lung cancer cells; the secretory leukoprotease inhibitor (SLPI) promoter, which is specific for SLPI-expressing carcinomas; the tyrosinase promoter, which is specific for melanoma cells; the stress inducible grp78/BiP promoter, which is specific for fibrosarcoma/tumorigenic cells; the AP2 adipose enhancer, which is specific for adipocytes; the a-1 antitrypsin transthyretin promoter, which is specific for hepatocytes; the interleukin-10 promoter, which is specific for glioblastoma multiform cells; the c-erbB-2 promoter, which is specific for pancreatic, breast, gastric, ovarian, and non-small cell lung cells; the a-B-crystallin/heat shock protein 27 promoter, which is specific for brain tumor cells; the basic fibroblast growth factor promoter, which is specific for glioma and meningioma cells; the epidermal growth factor receptor promoter, which is specific for squamous cell carcinoma, glioma, and breast tumor cells; the mucin-like glycoprotein (DF3, MUC1) promoter, which is specific for breast carcinoma cells; the mtsl promoter, which is specific for metastatic tumors; the NSE promoter, which is specific for small-cell lung cancer cells; the somatostatin receptor promoter, which is specific for small cell lung cancer cells; the c-erbB-3 and c-erbB-2 promoters, which are specific for breast cancer cells; the c-erbB4 promoter, which is specific for breast and gastric cancer; the thyroglobulin promoter, which is specific for thyroid carcinoma cells; the ofetoprotein (AFP) promoter, which is specific for hepatoma cells; the villin promoter, which is specific for gastric cancer cells; and the albumin promoter, which is specific for hepatoma cells. In another embodiment, the TERT promoter or survivin promoter are used.

For example, in some embodiments, heterologous nucleic acid sequences encoding the ATF3 protein (Gene ID: 467, NC_000001.11) are operably linked to a promoter, for example, a CMV promoter or an Egr promoter.

### Non-Viral Vectors

Any appropriate non-viral vectors can be used to introduce Atf3 gene into a tumor cell. Examples of non-viral vectors include, without limitation, vectors based on plasmid DNA or RNA, retroelements, transposons, and episomal vectors. In one embodiment, vectors are delivered to cells via nucleofection, a type of electroporation. In one embodiment, vectors are delivered to cells via colloidal dispersion systems that include, for example, macromolecule complexes, nanocapsules, microspheres, beads, and lipid-based systems including oil-in-water emulsions, micelles, mixed micelles, and liposomes.

Non-viral vectors can also be delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased by using dioleoylphosphatidylethanolamine during transduction. High efficiency liposomes are commercially available.

In one embodiment, the non-viral vector is an episomal vector. The episomal vector can include one or more pluripotency genes operatively linked to at least one regulatory sequence for expressing the factors. The episomal vectors of the invention can also include components allowing the vector to replicate in cells. For example, the Epstein Barr oriP/Nuclear Antigen-1 (EBNA-1) combination can support vector self-replication in mammalian cells, particularly primate cells. The EBNA1 trans element and OriP cis element derived from the EBV genome enables a simple plasmid to replicate and sustain as an episome in proliferating human cells.

The present inventions are not limited to the ATF3 activators exemplified in the above. A skilled person in the art can determine whether an agent meets the criteria set forth in its definition as provided above without undue efforts.

### Methods and Therapies

Another aspect of the invention is related to methods for treating an IR resistant tumor in a subject. The methods comprise administering to the subject in need thereof a therapeutically effective amount of an ATF3 activator. Further, the present invention provides a use of an ATF3 activator in the preparation of a pharmaceutical composition for the treatment of an IR resistant tumor in a subject. Yet further, the present invention provides use of an ATF3 activator for treatment of an IR resistant tumor in a subject. The disclosure also provides an ATF3 activator as described above for use in a method for treating symptoms involving an IR resistant tumor.

In certain embodiments, an ATF3 activator or a pharmaceutical composition comprising the ATF3 activator is administered parenterally, e.g. intravenously, intramuscularly, percutaneously or intracutaneously.

In some embodiments, it may be desirable to combine an ATF3 activator with other therapies for example chemo-therapeutic agents effective in the treatment of an IR resistant tumor. For example, the treatment of an IR resistant tumor may be implemented with an ATF3 activator and other anti-tumor therapies available in the market.

In certain embodiments, the methods of treating an IR resistant tumor prevent progression of the tumor and/or the onset of disease caused by the tumor. Thus, in some embodiments, a method for preventing the progression of an IR resistant tumor and/or the onset of disease caused by an IR resistant tumor, comprising administering of an effective amount of an ATF3 activator to a subject in need thereof is provided. In certain embodiments, the methods consist of treating an IR resistant tumor to prevent the onset, progression and/or recurrence of a symptom associated with the tumor. In certain embodiments, a method is provided for preventing a symptom associated with an IR resistant tumor in a subject, comprises administering an effective amount of an ATF3 activator to a subject in need thereof.

### Compositions

Yet another aspect of the present invention provides a pharmaceutical composition comprising a therapeutically effective amount of an ATF3 activator and a pharmaceutically acceptable carrier. The pharmaceutical composition is intended for treatment of an IR resistant tumor in a subject. The ATF3 activator may be prepared in a suitable pharmaceutically acceptable carrier or excipient. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologies standards.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### EXAMPLES

### Materials and Methods

Cells and virus. HCT116, D54, U251MG, T24, Widr and Du145 cell lines were obtained from American Type Culture Collection and cultured in DMEM medium supplemented with 10% FBS. The limited-passage HSV-1(F) is a prototype HSV-1 strain.

Antibodies. Mouse monoclonal antibodies against Flag and β-actin were purchased from Sigma-Aldrich. Mouse polyclonal antibody against ICP8 has been described elsewhere. Rabbit polyclonal antibody against ATF3 was purchased from Santa Cruz. All the antibodies were used at a dilution of 1:1,000.

Generation of ATF3 overexpression cell line using lentivirus. Du145 cells were seeded in 6-well plate 24 hours before infection. Cells were infected by lentivirus encoding a scrambled sequence or the Flag tagged coding sequence of ATF3 at the amino terminus in the medium of polybrene (8 μg/ml) for 30 hours. Cells were further selected by 1 μg/ml of puromycin for 3 days. The survived cells were collected and ATF3 protein bearing a FLAG tag was detected by immunoblot.

Generation of ATF3 depleted cell line using CRISPR. A CRISPR kit targeting the second exon of human ATF3 gene was obtained from Origene (Cat. No. KN202897). Parental HCT116 cells were cotransfected with a gRNA vector plasmid targeting sequences 5′-CTTCCTTGACAAAGGGCGTC-3′ (115th-134th bp in exon 2) or 5′-CCACCGGATGTCCTCTGCGC -3′ (169th-188th bp in exon2), and a donor vector containing a selection cassette expressing GFP-puromycin was inserted into the targeted site in human ATF3 gene. Selected cells (puromycin resistant) were serially diluted to form single cell-derived colonies. The colonies were further screened for expression of ATF3 by immunobloting. Clones 7 and 8 consisted largely of ATF3^{-/-} cells.

Immunoblot analyses. Cells were collected at the indicated times after irradiation or infection. Equal amounts of total proteins were electrophoretically separated on a 12.5% denaturing polyacrylamide gel, transferred to a nitrocellulose sheet, and probed with the mouse monoclonal antibody to β-actin, mouse polyclonal antibody to ICP8 and rabbit polyclonal antibodies to ATF3 listed above.

MTT Assay. Cell viability was determined by the 3-(4,5-dimethylthiazol-2-yl)-2,5-diphenyl tetrazolium bromide (MTT, Sigma, USA) assay. Briefly, cells were seeded in 96-well plates at 2,000 cells/well and incubated overnight, followed by treatment with mock or increasing amounts of IR. MTT solution (5 mg/ml in PBS, 20 μl/well) was added to the cells to produce formazan crystals. MTT solution was substituted by 150 μl DMSO 4 hours later to solubilize the formazan crystal. The optical absorbance was determined at 490 nm using an iMark microplate reader (Bio-Rad, USA).

Colony formation assay. Cells were seeded to form colonies in 6-well plates and treated the next day by increasing amounts of IR. When sufficiently large colonies with at least 50 cells were visible (12-15 d), the plates were fixed with methanol and stained with crystal violet. Colonies with more than 50 cells were counted, and the surviving fraction was calculated.

### Results

ATF3 is Induced in IR Sensitive Cell Lines but Not in IR Resistant Cell Lines Exposed to 6Gy.

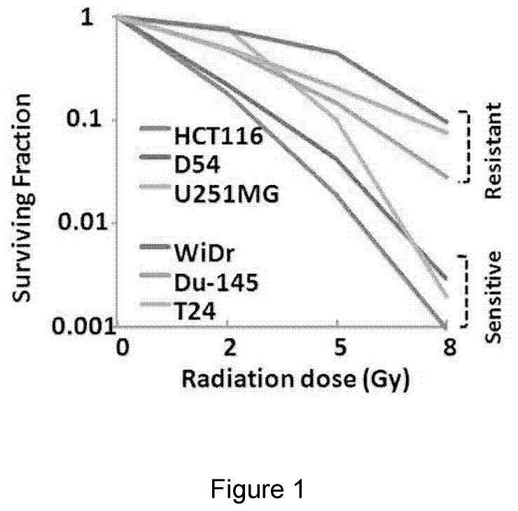

For the studies described in this report we tested 6 cell lines. Of the 6, Du145, Widr, T24 derived from prostate, colon and bladder respectively were reported as IR resistant. The other 3 cell lines, i.e. HCT116 (colon), D54 (CNS, central nervous system) and U251MG (CNS) were reported as sensitive to IR. The viability of the cells following exposure to different doses of radiation as measured by a colony formation assay is shown in FIG. 1.

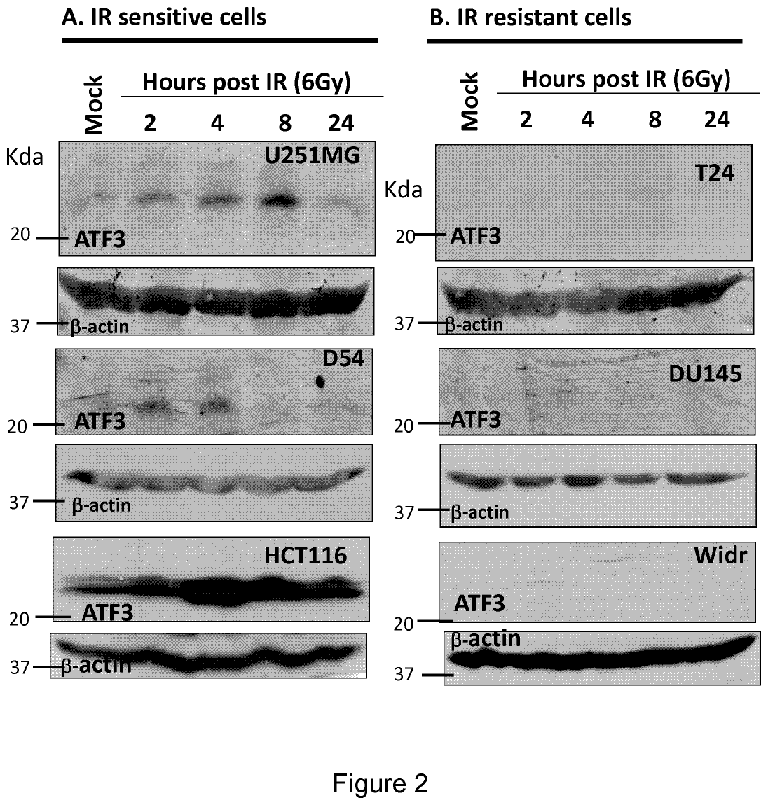

We next examined the 6 cell lines for production of ATF3 following administration of 6Gy. As shown in FIG. 2 Panel A all 3 IR sensitive cell lines expressed ATF3 at low levels constitutively but at increasing levels within hours after exposure to IR. In contrast, as illustrated in FIG. 2 Panel B, ATF3 was not detected in all IR resistant cell lines either before or after exposure to 6Gy.

Depletion of ATF3 Causes Sensitive Cells to Become Resistant to IR.

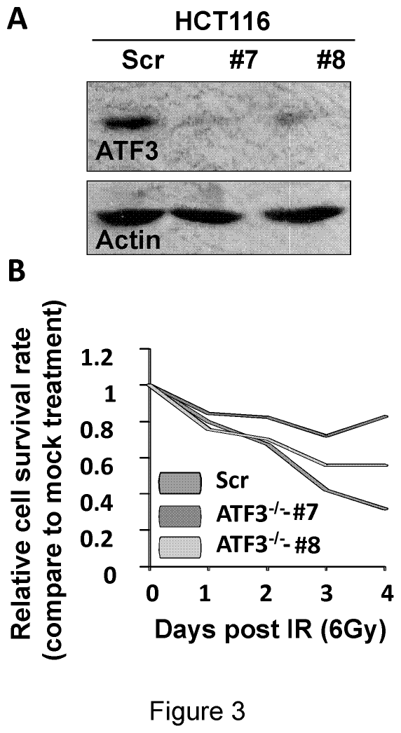

To test the effect of ATF3 depletion in IR sensitive cells we knocked out ATF3 from HCT116 cells as described in Materials and Methods and then selected for the studies described below 3 cell clones. Clone 1 designated as Scr was transfected with a scrambled sequence and served as a control. Clones 7 and 8 were only partially purified and consisted of mixtures of ATF3^{-/-} cells ATF3^{+/+} cells (FIG. 3 panel A). The 3 cell lines were subjected to 6Gy and monitored for viability using the MTT assay. The results show that the sensitivity to IR was inversely related the presence ATF3. Thus the Scr clone was the most sensitive whereas clone 7 containing the fewest ATF3^{+/+} cells was the most resistant. These results suggest that expression of ATF3 is essential for rendering cells sensitive to IR.

Stable Expression of ATF3 in Du145 Cells, an IR Resistant Cell Line, Results in Cell Death in the Absence of Exposure to IR.

In this series of experiments we first transformed Du145 cells with the lentivirus encoding a Flag-tagged ATF3 or the lentivirus in which the nucleotide sequences encoding ATF3 were scrambled. The cultures were seeded in 6 well plates and monitored daily. The results were as follows:

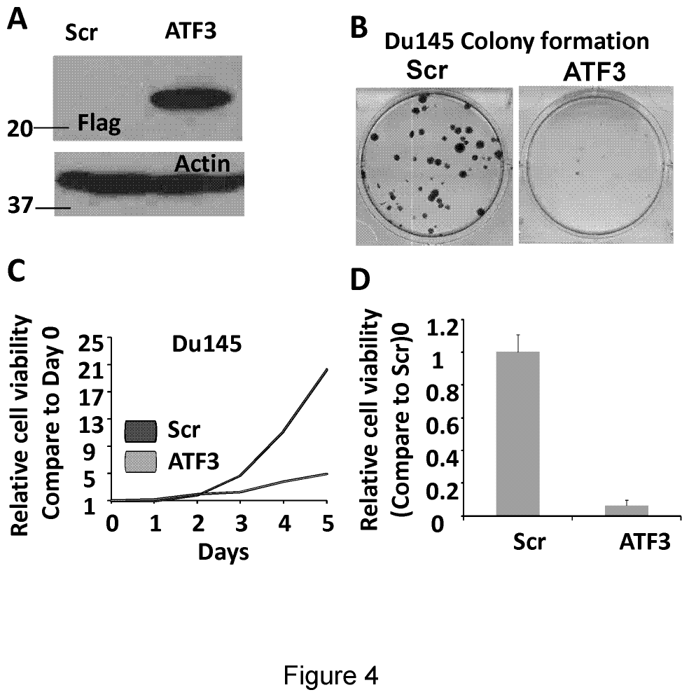

(i) FIG. 4 panel A shows that cells transformed with the lentivirus encoding a Flag-tagged ATF3 but not the cells transformed with a scrambled sequence stably expressed ATF3.

(ii) FIG. 4 Panel B shows the appearance of colonies of Du145 cells 14 days after seeding of transformed cells onto 6 well plates. The decrease in the population of Du145 cells transformed with ATF3 is mirrored by the relative cell viability (MTT assay) as shown in panel C and the relative fraction of cells surviving transformation by a lentivirus encoding ATF3 (Day 14 after seeding, Panel D).

IR Resistant Cell Lines Du145 and Widr Retained the Ability to Express ATF3 Following Stimulation by Means Other Than IR.

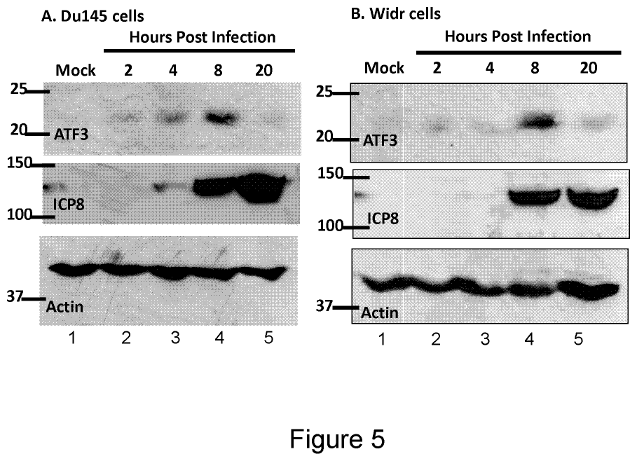

The objective of this experiment was to test the hypothesis that in cells resistant to IR the pathway to activation of PKR by IR is impaired but that ATF3 can be induced in response to other forms of stress. Previous studies have shown that ATF3 expression is activated in response to HSV-1 infection. To test our hypothesis, we monitored the accumulation of ATF3 in Du145 as well as Widr cells exposed to 10 PFU of HSV-1(F) per cells. In addition to ATF3 we monitored the accumulation of ICP8, a viral protein as an indicator that the cells were indeed infected and produced viral gene products. The results shown in FIG. 5 indicate that the Du145 and Widr infected cells responded to infection by production of ATF3. We conclude that the IR resistant cells retain the capacity to express ATF3.

The major findings of the present invention are two fold: First, ATF3 is an effector of cell death. It follows that depletion of ATF3 causes IR sensitive cells to become resistant to IR. Second, IR resistant cells do not express ATF3. Forced expression of ATF3 by infection of a lentivirus encoding the protein results in the death of IR resistant cells.

The second key finding is evidence that IR resistant cells are able to respond to stress by producing ATF3 in response to stress induced by means other than IR. The results suggest that (a) ATF3 is activated by multiple pathways, and (b) In IR resistant cells the pathway to activation of ATF3 is ineffective as a consequence of a selective mutation but that ATF3 remains inducible by other means.

As noted above ATF3 is made in response to stress mediated by a variety of receptors. Loss of a component of the pathway leading from the receptor of IR stress would lead to development of resistance to IR. Since the resistant cells respond to stress of infection by making ATF3, it would be predicted that ATF3 could be induced in patients subjected to radiotherapy by small molecules, exosome carrying non coding RNAs, etc. The results of the present invention predict that the synthesis of ATF3 would result in the death of IR resistant cells but that this procedure will not affect IR sensitive cells since they would respond to IR by making ATF3.

It should be stressed that activated ATF3 is not a radiosensitizer: ATF3 defines the fate of stressed cells but in many instance, particularly in neuronal cells it activates a protective pathway rather than one that leads to cell death. The data clearly indicate that in both IR resistant and sensitive cells it plays an essential role as an IR cell death activator (IRCDA) and should be defined as such.

It should be understood that although the present invention has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein. In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. The disclosures illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

1. A method for treatment of an ionizing radiation resistant tumor in a subject, comprising administering to the subject in need of treatment a therapeutically effective amount of an ATF3 activator.

2. The method of claim 1, wherein the tumor does not express ATF3 protein.

3. The method of claim 2, wherein the tumor retains capability to express ATF3 protein.

4. The method of claim 1, wherein the ATF3 activator is selected from a group consisting of a small molecule capable to induce ATF3 expression, a vector carrying a polynucleotide encoding ATF3 protein, and a vector carrying a non-coding RNA directing to an ATF3 expression inhibitor.

5. The method of claim 4, wherein the small molecule capable to induce ATF3 expression is selected from a group consisting of curcumin, non-steroidal anti-inflammatory drugs, progesterone, a phosphatidylinositol inhibitor, tert-butylhydroquinone (tBHQ) and butylated hydroxyanisole (BHA).

6. The method of claim 4, wherein the vector carrying a polynucleotide encoding ATF3 protein is a viral or non-viral vector carrying an ATF3 protein encoding DNA.

7. The method of claim 6, wherein the viral vector is a retrovirus, adenovirus, adeno-associated virus, herpes simplex virus, vaccinia virus or baculovirus.

8. The method of claim 4, wherein the non-coding RNA directing to an ATF3 expression inhibitor is an antisense oligomer.

9. The method of claim 8, wherein the antisense oligomer is selected from dsRNA, siRNA, and shRNA.

10. The method of claim 4, wherein the vector carrying a non-coding RNA is an exosome.

11. The method of claim 1, wherein the administration of the ATF3 activator results in expression of the ATF3 protein in a cell of the tumor.

12. The method of claim 1, wherein the administration of the ATF3 activator leads to (a) an elevated expression of ATF3 in a cell of the tumor, (b) accumulation of ATF3 protein in a cell of the tumor, or (c) tumor cell death.

13. (canceled)

14. (canceled)

15. The method of claim 1, wherein the subject is a mammal.

16. The method of claim 15, wherein the mammal is a human being.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. An artificial vector for treatment of an ionizing radiation resistant tumor in a subject, wherein the vector carries a polynucleotide encoding ATF3 protein.

30. The artificial vector of claim 29, wherein the polynucleotide is a DNA or RNA sequence encoding ATF3 protein.

31. The artificial vector of claim 29, wherein the artificial vector is a recombinant virus comprising a polynucleotide encoding ATF3 protein.

32. The artificial vector of claim 31, wherein the recombinant virus is a retrovirus, adenovirus, adeno-associated virus, herpes simplex virus, vaccinia virus or baculovirus.

33. The artificial vector of claim 29, wherein the tumor does not express ATF3 protein.

34. The artificial vector of claim 33, wherein the tumor retains capability to express ATF3 protein.

35. The artificial vector of claim 29, wherein upon introduction of the artificial vector into the cell (a) the ATF3 protein is stably expressed in the tumor cell, or (b) the ATF3 protein is accumulated in the tumor cell.

36. (canceled)

37. A pharmaceutical composition comprising the artificial vector of claim 29, and a pharmaceutically acceptable excipient.
